(19) United States
(12) Patent Application Publication
(10) Pub. No.: US 2014/0140991 A1
(43) Pub. Date: May 22, 2014

(54) METHODS OF TREATING A DISEASE OR DISORDER ASSOCIATED WITH BRUTON'S TYROSINE KINASE

(71) Applicant: Celgene Avilomics Research, Inc., Bedford, MA (US)

(72) Inventors: Tom Daniel, LaJolla, CA (US); Kenichi Takeshita, New York, NY (US); Kenneth Foon, Summit, NJ (US); Jay Mei, North Wales, PA (US)

(73) Assignee: Celgene Avilomics Research, Inc., Bedford, MA (US)

(21) Appl. No.: 14/084,123

(22) Filed: Nov. 19, 2013

Related U.S. Application Data
(60) Provisional application No. 61/728,701, filed on Nov. 20, 2012.

(51) Int. Cl. A61K 39/395; A61K 31/185; A61K 31/505

(52) U.S. Cl. CPC A61K 39/3955; A61K 31/505; A61K 31/185
USPC 424/133.1

(57) ABSTRACT

The present invention provides methods of treating, stabilizing or lessening the severity or progression of a disease or disorder associated with BTK.

### CROSS REFERENCE TO RELATED APPLICATIONS

The present application claims priority to U.S. provisional application No. 61/728,701, filed Nov. 20, 2012, the entirety of which is hereby incorporated by reference.

### FIELD OF THE INVENTION

The present invention provides methods of treating, stabilizing or lessening the severity or progression of a disease or disorder associated with Bruton's Tyrosine Kinase (“BTK”).

### BACKGROUND OF THE INVENTION

The search for new therapeutic agents has been greatly aided in recent years by a better understanding of the structure of enzymes and other biomolecules associated with diseases. One important class of enzymes that has been the subject of extensive study is protein kinases.

Protein kinases constitute a large family of structurally related enzymes that are responsible for the control of a variety of signal transduction processes within the cell. Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.).

In general, protein kinases mediate intracellular signaling by effecting a phosphoryl transfer from a nucleoside triphosphate to a protein acceptor that is involved in a signaling pathway. These phosphorylation events act as molecular on/off switches that can modulate or regulate the target protein biological function. These phosphorylation events are ultimately triggered in response to a variety of extracellular and other stimuli. Examples of such stimuli include environmental and chemical stress signals (e.g., osmotic shock, heat shock, ultraviolet radiation, bacterial endotoxin, and H₂O₂), cytokines (e.g., interleukin-1 (IL-1) and tumor necrosis factor α (TNF-α)), and growth factors (e.g., granulocyte macrophage-colony-stimulating factor (GM-CSF), and fibroblast growth factor (FGF)). An extracellular stimulus may affect one or more cellular responses related to cell growth, migration, differentiation, secretion of hormones, activation of transcription factors, glucose metabolism, control of protein synthesis, and regulation of the cell cycle.

Many diseases are associated with abnormal cellular responses triggered by protein kinase-mediated events as described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases. Accordingly, there remains a need to find protein kinase inhibitors useful as therapeutic agents.

### SUMMARY OF THE INVENTION

Chronic lymphocytic leukemia (CLL) is a lymphoproliferative malignancy characterized by progressive accumulation of morphologically mature but functionally incompetent lymphocytes in the blood, bone marrow, and lymphoid tissues. It affects mainly elderly individuals with the median age at presentation of 65 to 70 years. Small lymphocytic lymphoma (SLL) and CLL are generally considered a different manifestation of the same disease. While CLL is found in the blood and bone marrow, SLL presents primarily in the lymph nodes. The clinical course of CLL/SLL ranges from indolent disease with long-term survival over 12 years to aggressive disease with median survival of 2 years. The average age of diagnosis with CLL/SLL is approximately 60 years.

Despite newly approved therapeutic agents and combination therapies, CLL/SLL remains an incurable disease and most patients eventually relapse and/or die. Improved and novel combination treatments for subjects with CLL/SLL requiring treatment remain an unmet medical need.

Bruton's tyrosine kinase (Btk) is a non-receptor tyrosine kinase with restricted cellular expression largely limited to B-lymphocytes, monocytes, and mast cells or basophils. Btk is a critical component of the B-cell receptor (BCR) signaling network and is crucial for B-cell development. Investigation has revealed that some B-cell malignancies, including diseases such as CLL/SLL, depend on BCR signaling, suggesting that interruption of such signaling could be a promising therapeutic opportunity. Recently, clinical anti-tumor responses in various B-cell non-Hodgkin's Lymphoma (NHL) and CLL/SLL have been reported with agents that inhibit spleen tyrosine kinase (Syk) and Btk, both components of the BCR signaling pathway.

United States published patent application number US 2010/0029610, published Feb. 4, 2010 (“the '610 publication,” the entirety of which is hereby incorporated herein by reference), describes certain 2,4-disubstituted pyrimidine compounds which covalently and irreversibly inhibit activity of one or more protein kinases, including BTK, a member of TEC-kinases. Such compounds include N-(3-(5-fluoro-2-(4-(2-methoxyethoxy)phenylamino)pyrimidin-4-ylamino)phenyl)acrylamide, hereinafter referred to as Compound 1, which is designated as compound number I-182 in the '610 publication. The synthesis of Compound 1 is described in detail at Example 20 of the '610 publication Compound 1 is active in a variety of assays and therapeutic models demonstrating covalent, irreversible inhibition of BTK (in enzymatic and cellular assays). Notably, Compound 1 is a potent, selective, orally available, small molecule which was found to inhibit B-cell proliferation and activation. Compound 1 is therefore useful for treating one or more disorders associated with activity of BTK.

Accordingly, among other things, the present invention provides methods of treating, stabilizing or lessening the severity or progression of one or more diseases and conditions associated with BTK. In some aspects, the present invention provides methods of treating, stabilizing or lessening the severity or progression of one or more diseases and conditions associated with BTK comprising administering to a patient in need thereof a pharmaceutically acceptable composition comprising N-(3-(5-fluoro-2-(4-(2-methoxyethoxy)phenylamino)pyrimidin-4-ylamino)phenyl)acrylamide (1):

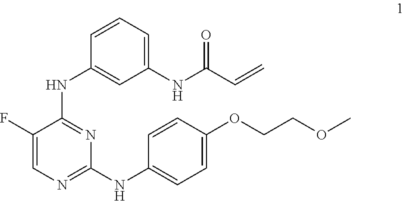 [SMILES: C=CC(=O)Nc1cccc(Nc2nc(Nc3ccc(OCCOC)cc3)ncc2F)c1]

In some embodiments, the present invention provides a method of treating, stabilizing or lessening the severity or progression of one or more diseases and conditions associated with BTK comprising administering to a patient in need thereof Compound 1 in combination with rituximab.

In some embodiments, the present invention provides a method of treating, stabilizing or lessening the severity or progression of one or more diseases and conditions associated with BTK comprising administering to a patient in need thereof a composition comprising Compound 1 in combination with a composition comprising rituximab.

In some embodiments, provided methods comprise administering to a patient in need thereof Compound 1 in combination with rituximab, wherein Compound 1 is administered once a day. In some embodiments, provided methods comprise administering to a patient in need thereof Compound 1 in combination with rituximab, wherein Compound 1 is administered twice a day. In some such embodiments, rituximab is administered once during a 28-day cycle. Accordingly, in some embodiments, provided methods comprise administering to a patient in need thereof Compound 1 in combination with rituximab, wherein Compound 1 is administered twice a day and rituximab is administered once during a 28-day cycle.

In some embodiments, the provided methods comprising administering to a patient in need thereof a composition comprising Compound 1 and rituximab.

In some embodiments, the disease or condition associated with BTK is selected from chronic lymphocytic leukemia and small lymphocytic lymphoma.

In some embodiments, the present invention provides a method of treating, stabilizing or lessening the severity or progression of chronic lymphocytic leukemia (CLL), the method comprising administering to a patient in need thereof Compound 1 in combination with rituximab.

In some embodiments, the present invention provides a method of treating, stabilizing or lessening the severity or progression of small lymphocytic lymphoma (SLL), the method comprising administering to a patient in need thereof Compound 1 in combination with rituximab.

In some embodiments, provided therapies comprise orally administering to a patient Compound 1 in combination with rituximab. In some embodiments, each of Compound 1 and rituximab is administered in the form of a pharmaceutical formulation. In some embodiments, the pharmaceutical formulation comprising Compound 1 is a capsule formulation. In some embodiments, the pharmaceutical formulation comprising rituximab is an intravenous (IV) formulation.

In some embodiments, the present invention also provides dosing regimens and protocols for administering to patients in need thereof Compound 1 in combination with rituximab. Such methods, dosing regimens and protocols for the administration of said combination are described in further detail, below.

### DETAILED DESCRIPTION OF THE INVENTION

### Definitions

As used herein, the term “antibody”, or grammatical variations thereof (i.e., antibodies), refers to polypeptide(s) capable of binding to an epitope. In some embodiments, an antibody is a full-length antibody. In some embodiments, an antibody is less than full length (i.e., an antibody fragment) but includes at least one binding site. In some such embodiments, the binding site comprises at least one, and preferably at least two sequences with structure of antibody variable regions. In some embodiments, the term “antibody” encompasses any protein having a binding domain which is homologous or largely homologous to an immunoglobulin-binding domain. In particular embodiments, the term “antibody” encompasses polypeptides having a binding domain that shows at least 99% identity with an immunoglobulin-binding domain. In some embodiments, the antibody is any protein having a binding domain that shows at least 70%, at least 80%, at least 85%, at least 90% or at least 95% identity with an immunoglobulin-binding domain. Antibody polypeptides in accordance with the present invention may be prepared by any available means, including, for example, isolation from a natural source or antibody library, recombinant production in or with a host system, chemical synthesis, etc., or combinations thereof. In some embodiments, an antibody is monoclonal or polyclonal. In some embodiments, an antibody may be a member of any immunoglobulin class, including any of the human classes IgG, IgM, IgA, IgD and IgE. In certain embodiments, an antibody is a member of the IgG immunoglobulin class. In some embodiments, the term “antibody” refers to any derivative of an antibody that possesses the ability to bind to an epitope of interest. In some embodiments, an antibody fragment comprises multiple chains that are linked together, for example, by disulfide linkages. In some embodiments, an antibody is a human antibody. In some embodiments, an antibody is a humanized antibody. In some embodiments, humanized antibodies include chimeric immunoglobulins, immunoglobulin chains or antibody fragments (Fv, Fab, Fab′, F(ab′)₂ or other antigen binding subsequences of antibodies) that contain minimal sequence derived from non-human immunoglobulin. In some embodiments, humanized antibodies are human immunoglobulin (recipient antibody) in which residues from a complementary-determining region (CDR) of the recipient are replaced by residues from a CDR of a non-human species (donor antibody) such as mouse, rat or rabbit having the desired specificity, affinity and capacity. In particular embodiments, antibodies for use in the present invention bind to particular epitopes of CD20. In some embodiments, epitopes of CD20 to which anti-CD20 antibodies bind include, for example, ¹⁷⁰ANPS¹⁷³ (Binder et al., Blood 2006, 108(6): 1975-1978), FMC7 (Deans et al., Blood 2008, 111(4): 2492), Rp5-L and Rp15-C (mimotopes of CD20) (Perosa et al., J. Immunol. 2009, 182:416-423), ¹⁸²YCYSI¹⁸⁵ (Binder et al., Blood 2006, 108(6): 1975-1978) and WEWTI (a mimic of ¹⁸²YCYSI¹⁸⁵) (Binder et al., Blood 2006, 108(6): 1975-1978). In some embodiments, an anti-CD20 antibody has a binding affinity (K_{d}) for an epitope of CD20 of less than 12 nM, less than 11 nM, less than 10 nM, less than 9 nM, less than 8 nM, less than 7 nM, less than 6 nM, less than 5 nM, less than 4 nM, less than 3 nM, less than 2 nM or less than 1 nM.

As used herein, the term “biosimilar” (for example, of an approved reference product/biological drug, such as a protein therapeutic, antibody, etc.) refers to a biologic product that is similar to the reference product based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is approved and intended to be used and for which approval is sought (e.g., that there are no clinically meaningful differences between the biological product and the reference product in terms of the safety, purity, and potency of the product).

In some embodiments, the biosimilar biological product and reference product utilizes the same mechanism or mechanisms of action for the condition or conditions of use prescribed, recommended, or suggested in the proposed labeling, but only to the extent the mechanism or mechanisms of action are known for the reference product. In some embodiments, the condition or conditions of use prescribed, recommended, or suggested in the labeling proposed for the biological product have been previously approved for the reference product. In some embodiments, the route of administration, the dosage form, and/or the strength of the biological product are the same as those of the reference product. In some embodiments, the facility in which the biological product is manufactured, processed, packed, or held meets standards designed to assure that the biological product continues to be safe, pure, and potent. The reference product may be approved in at least one of the U.S., Europe, or Japan. A biosimilar can be for example, a presently known antibody having the same primary amino acid sequence as a marketed antibody, but may be made in different cell types or by different production, purification or formulation methods.

As used herein, the terms “combination”, “in combination with” or “combination therapy” refer to those situations in which two or more different pharmaceutical agents are administered in overlapping regimens so that the subject is simultaneously exposed to both agents. In some embodiments, such combinations refer to simultaneously administering to a subject separate dosage forms of Compound 1 and rituximab. In some embodiments, such combinations refer to contemporaneously administering to a subject separate dosage forms of Compound 1 and rituximab, wherein Compound 1 is administered before, during or after administration of rituximab. In some embodiments, simultaneous or contemporaneous exposure of Compound 1 and rituximab is effected via different dosage regimens appropriate for each therapeutic agent. For example, Compound 1 may be administered once or twice daily for one or more 28-day cycles, whereas rituximab may be administered once during a 28-day cycle.

As used herein, a “disease or disorder associated with BTK” or a “BTK-mediated disorder” means any disease or other deleterious condition in which BTK, or a mutant thereof, is known or suspected to play a role. Accordingly, another embodiment of the present invention relates to preventing, treating, stabilizing or lessening the severity or progression of one or more diseases in which BTK, or a mutant thereof, is known or suspected to play a role. Specifically, the present invention relates to a method of treating or lessening the severity of a proliferative disorder, wherein said method comprises administering to a patient in need thereof Compound 1 in combination with rituximab.

The term “refractory CLL/SLL” as used herein is defined as CLL/SLL which was treated with at least one line of prior therapy (i) without achieving at least a partial response to therapy or (ii) which progressed within 6 months of treatment.

The term “relapsed CLL/SLL” as used herein is defined as CLL/SLL which progressed after ≧6 months post-treatment after achieving partial response or complete response to therapy.

The term “subject”, as used herein, means a mammal and includes human and animal subjects, such as domestic animals (e.g., horses, dogs, cats, etc.).

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a “therapeutically effective amount” is at least a minimal amount of a compound, or composition containing a compound, which is sufficient for treating one or more symptoms of a disorder or condition associated with Bruton's tyrosine kinase.

The terms “treat” or “treating,” as used herein, refers to partially or completely alleviating, inhibiting, delaying onset of, preventing, ameliorating and/or relieving a disorder or condition, or one or more symptoms of the disorder or condition. As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The expression “unit dosage form” as used herein refers to a physically discrete unit of therapeutic formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

### Compound 1 is an Irreversible BTK Inhibitor

As described above, Bruton's tyrosine kinase (Btk) is a non-receptor tyrosine kinase with restricted cellular expression largely limited to B-lymphocytes, monocytes, and mast cells or basophils. Btk is a critical component of the B-cell receptor (BCR) signaling network and is crucial for B-cell development. Investigation has revealed that some B-cell malignancies, including CLL/SLL, depend on BCR signaling, suggesting that interruption of such signaling could be a promising therapeutic opportunity. Recently, clinical anti-tumor responses in various B-cell non-Hodgkin's lymphoma (NHL) and CLL/SLL have been reported with agents that inhibit spleen tyrosine kinase (Syk) and Btk, both components of the BCR signaling pathway.

Compound 1 is active in a variety of assays and therapeutic models demonstrating covalent, irreversible inhibition of BTK (in enzymatic and cellular assays). Compound 1 inhibits Btk activity by binding with high affinity to the adenosine triphosphate (ATP) binding site of Btk and forming a targeted covalent bond with the Btk protein, providing rapid, complete, and prolonged inhibition of Btk activity, both in vitro and in vivo.

Phosphorylation of the auto-phosphorylation site on Btk (Tyr223) and the Btk responsive site (Tyr1217) on PLCγ2 in Ramos cells, a human Burkitt lymphoma cell line, was inhibited by Compound 1 with an effective concentration required for 50% inhibition (EC₅₀) of 1 nM to10 nM. Compound 1 demonstrates a high degree of selectivity in cellular assay systems against related kinases.

In single dose studies in healthy subjects, Compound 1 evidenced adequate safety, predictable pharmacokinetics (PK), and, at doses greater than 0.5 mg/kg, 80% to 100% occupancy of the Btk receptor target in normal human peripheral blood B-cells. A phase I dose escalation study of a single agent of Compound 1 is currently being conducted in different hematologic malignancies, including CLL/SLL.

### Anti-CD20 Antibodies

CD20, the first B-cell specific antigen defined by the monoclonal antibody tositumomab, plays a critical role in B-cell development. Human CD20 is a 297 amino acid (30- to 35-kDa) phosphoprotein with four transmembrane domains encoded by the gene MS4A1 located on chromosome 11q12.2. CD20 plays a critical role in B-cell development and is a biomarker for immunotherapies targeting B-cell derived diseases. CD20 is an integral membrane protein expressed by B lymphocytes in early stages of differentiation and by most B cell lymphomas, but not by differentiated plasma cells. CD20 remains on the membrane of B cells without dissociation or internalization upon antibody binding. CD20 functions though binding to the Src family of tyrosine kinases, such as Lyn, Fyn and Lck, and believed to be involved as a result in the phosphorylation cascade of intracellular proteins. Anti-CD20 antibodies are broadly classified into type I and type II antibodies. Both types of anti-CD 20 antibodies exhibit equal ability in activating Fc-FcγR interactions such as antibody-dependent cellular cytotoxicity (ADCC) and phagocytosis. Type I anti-CD20 antibodies redistribute CD20 into membrane lipid rafts and potently activate complement-dependent cytotoxicity (CDC). Type II anti-CD20 antibodies weakly activate CDC but more potently induce direct programmed cell death.

In some embodiments, the present invention encompasses the recognition that the combination of a BTK inhibitor, i.e. Compound 1, in combination with an anti-CD20 antibody is useful in treating BTK-mediated diseases or disorders. Accordingly, in some embodiments, the present invention comprises a method of treating a BTK-mediated disease or disorder, the method comprising administering to a patient in need thereof Compound 1 in combination with a CD20 antibody. A person of ordinary skill in the art can readily identify and select additional anti-CD20 antibodies that are useful in the present invention. For example, in some embodiments, such antibodies are described, for example, in U.S. Pat. Nos. 8,153,125, 8,147,832, 8,101,179, 8,084,582, 8,057,793 and 7,879,984, and U.S. Patent Publication Nos. 2011/0129412, 2012/0183545, 2012/0134990 and 2012/0034185.

In some embodiments, an anti-CD20 antibody for use in the present invention is a type I antibody. In some embodiments, an anti-CD20 for use in the present invention is a type II antibody.

In some embodiments, an anti-CD20 antibody is an antibody that binds to a CD20 epitope selected from ¹⁷⁰ANPS¹⁷³ and ¹⁸²YCYSI¹⁸⁵.

In some embodiments, an anti-CD20 antibody has a binding affinity (K_{d}) for an epitope of CD20 of less than 12 nM, less than 11 nM, less than 10 nM, less than 9 nM, less than 8 nM, less than 7 nM, less than 6 nM, less than 5 nM, less than 4 nM, less than 3 nM, less than 2 nM or less than 1 nM.

Rituximab is but one example of an anti-CD20 antibody; For ease of reference, provided methods and regimens detailed herein refer to an exemplary anti-CD20 antibody (i.e., rituximab); however, such reference is not intended to limit the present invention to a single anti-CD20 antibody. Indeed, all references to rituximab, or a biosimilar thereof, are to be read by a person skilled in the art to encompass the class of anti-CD20 antibodies. Accordingly, in some embodiments, the term “rituximab” encompasses all corresponding anti-CD20 antibodies that fulfill the requirements necessary for obtaining a marketing authorization as an identical or biosimilar product in a country or territory selected from the group of countries consisting of the USA, Europe and Japan.

In some embodiments, an anti-CD20 antibody has the same or similar activity as rituximab, or a biosimilar thereof. In some embodiments, an anti-CD20 antibody binds to the same or similar region or epitope as rituximab or a fragment thereof. In some embodiments, an anti-CD20 antibody competes with the binding of rituximab or a fragment thereof to CD20. In some embodiments, an anti-CD20 antibody is bioequivalent to rituximab or a fragment thereof. In some embodiments, an anti-CD20 antibody is a biosimilar of rituximab or a fragment thereof. In some embodiments, an anti-CD20 antibody is a variant or derivative of rituximab, including functional fragments, derivatives, or antibody conjugates.

### Rituximab

Rituximab (Rituxan® or MabThera®) is a genetically engineered cytolytic, chimeric murine/human monoclonal IgG₁ kappa antibody directed against the CD20 cell-surface molecule present in normal B lymphocytes and B-cell CLL and in most forms of non-Hodgkin's B-cell lymphomas. Rituximab has a binding affinity for the CD20 antigen of approximately 8.0 nM. Rituximab can induce complement-dependent cellular cytotoxicity (CDC) and anti-body-dependent cellular cytotoxicity (ADCC), leading to its clinical activity against lymphoma cells. Rituximab can also lead to apoptosis of B cells upon binding to CD20, thereby leading to direct inhibition of cellular growth.

Rituximab is produced by mammalian cell (Chinese Hamster Ovary) suspension culture in a nutrient medium containing the antibiotic gentamicin. Gentamicin is not detectable in the final product. Rituximab is a sterile, clear, colorless, preservative-free liquid concentrate for intravenous administration. Rituximab is supplied at a concentration of 10 mg/mL in either 100 mg/10 mL or 500 mg/50 mL single-use vials. Rituximab is formulated in polysorbate 80 (0.7 mg/mL), sodium citrate dihydrate (7.35 mg/mL), sodium chloride (9 mg/mL) and water for injection. The pH of Rituxan® (or MabThera®) is 6.5

Rituximab has been investigated in clinical studies and approved for treatment of patients with CLL in combination with fludarabine and cyclophosphamide, as well as patients with rheumatoid arthritis in combination with methotrexate. Rituximab is also approved for treatment of non-Hodgkin's lymphoma, Wegener's Granulomatosis and Microscopic Polyangiitis.

In some embodiments, provided methods comprise administering to a patient in need thereof a combination of Compound 1 and rituximab, wherein the patient is further treated with fludarabine and cyclophosphamide in accordance with the approved indications.

### I. General Dosing Protocol

In some embodiments, the present invention provides methods for treating, stabilizing or lessening the severity or progression of one or more diseases or conditions associated with BTK. In some embodiments, the present invention provides methods for preventing the progression of a disease or disorder associated with BTK. In some embodiments, the disease or disorder associated with BTK is selected from chronic lymphocytic leukemia (CLL) and small lymphocytic lymphoma (SLL).

In some embodiments, the disease or disorder associated with BTK is refractory CLL. In some embodiments, the disease or disorder associated with BTK is relapsed CLL. In some embodiments, the disease or disorder associated with BTK is refractory SLL. In some embodiments, the disease or disorder associated with BTK is relapsed SLL.

In some embodiments, provided methods comprise administering to a patient in need thereof Compound 1 in combination with rituximab. In some such embodiments, each of Compound 1 and rituximab is administered as a composition further comprising one or more pharmaceutically acceptable excipients.

In some embodiments, provided methods comprise administering to a patient in need thereof a therapeutically effective amount of Compound 1 in combination with a therapeutically effective amount of rituximab. Accordingly, in some embodiments, the present invention provides a method of treating, stabilizing or lessening the severity or progression of one or more diseases associated with BTK, the method comprising administering to a patient in need thereof a therapeutically effective amount of Compound 1 in combination with a therapeutically effective amount of rituximab.

In some embodiments, provided methods comprise administering Compound 1 in combination with rituximab, wherein Compound 1 is administered once daily (“QD”). In some embodiments, provided methods comprise administering Compound 1 in combination with rituximab, wherein Compound 1 is administered twice daily (“BID”). For purposes of clarity, administration of a 375 mg dose of Compound 1 “BID” means that the patient is administered two separate doses of 375 mg in one day.

In some embodiments, provided methods comprise administering Compound 1 in combination with rituximab, wherein rituximab is administered once during a 28-day cycle. In some embodiments, provided methods comprise administering Compound 1 in combination with rituximab, wherein rituximab is administered on cycle 1 day 2. In some embodiments, provided methods comprise administering Compound 1 in combination with rituximab, wherein rituximab is administered on day 1 of a 28-day cycle. In some such embodiments, rituximab is administered on day 1 of cycles 2-6. In some embodiments, rituximab is administered on day 1 of cycles 2-5. In some embodiments, rituximab is administered on day 1 of cycles 2-4. In some embodiments, rituximab is administered on day 1 of cycles 2-3.

In some embodiments, provided methods comprise administering Compound 1 in combination with rituximab, wherein Compound 1 is administered twice daily and rituximab is administered once during a 28-day cycle. In some embodiments, provided methods comprise administering Compound 1 in combination with rituximab, wherein Compound 1 is administered twice daily and rituximab is administered on cycle 1 day 2. In some embodiments, provided methods comprise administering Compound 1 in combination with rituximab, wherein Compound 1 is administered twice daily and rituximab is administered on day 1 of a 28-day cycle. In some such embodiments, rituximab is administered on day 1 of cycles 2-6.

In some embodiments, each of Compound 1 and rituximab is administered as pharmaceutically acceptable compositions. In some embodiments, a pharmaceutically acceptable composition comprising Compound 1 is formulated as an oral dosage form. In some embodiments, such oral dosage forms are capsules. In some embodiments, the pharmaceutically acceptable composition comprising rituximab is formulated as an intravenous composition.

In some embodiments, a pharmaceutically acceptable composition comprising Compound 1 comprises from about 5% to about 60% of Compound 1, or a pharmaceutically acceptable salt thereof, based upon total weight of the composition. In some embodiments, a pharmaceutically acceptable composition comprising Compound 1 comprises from about 5% to about 15% or about 7% to about 15% or about 7% to about 10% or about 9% to about 12% of Compound 1, based upon total weight of the composition. In some embodiments, provided methods comprise administering to a patient in need thereof a pharmaceutically acceptable composition comprising from about 25% to about 75% or about 30% to about 60% or about 40% to about 50% or about 40% to about 45% of Compound 1, based upon total weight of the formulation. In certain embodiments, provided regimens comprise administering to a patient in need thereof a pharmaceutically acceptable composition comprising from about 6%, about 7%, about 8%, about 9%, about 10%, about 11%, about 12%, about 13%, about 20%, about 30%, about 40%, about 41%, about 42%, about 43%, about 44%, about 45%, about 50%, about 60%, about 70%, or about 75% of Compound 1, based upon total weight of given composition or formulation.

Rituximab is commercially available as a 10 mg/mL solution comprising sodium citrate, polysorbate 80, sodium chloride, sodium hydroxide, hydrochloric acid and water. Commercially available vials comprise either 100 mg/10 mL or 500 mg/50 mL.

In some embodiments, a pharmaceutically acceptable composition comprises from about 1 mg/mL to about 4 mg/mL rituximab. In some embodiments, a pharmaceutically acceptable composition comprises from about 1 mg/mL, about 2 mg/mL, about 3 mg/mL or about 4 mg/mL rituximab. In some embodiments, a pharmaceutically acceptable composition comprises 10 mg/mL.

In some embodiments, provided methods comprise administering Compound 1 in combination with rituximab daily for a period of 1, 2, 3, 4, 5, 6, 7, 8, 9, 10, 11, 12, 13, 14, 15, 16, 17, 18, 19, 20, 21, 22, 23, 24, 25, 26, 27 or 28 days. In some embodiments, a treatment regimen comprises at least one 28-day cycle. As used herein, the term “28-day cycle” means that provided treatment regimens are administered to a patient in need thereof for 28 consecutive days. In some embodiments, the combination of Compound 1 and rituximab is administered for at least two, at least three, at least four, at least five or at least six 28-day cycles. In some embodiments, the combination of Compound 1 and rituximab is administered for at least seven, at least eight, at least nine, at least ten, at least eleven or at least twelve 28-day cycles. In some embodiments, the combination of Compound 1 and rituximab is administered for at least thirteen, at least fourteen, at least fifteen, at least sixteen, at least seventeen, at least eighteen, at least nineteen or at least twenty 28-day cycles. In some embodiments, the combination of Compound 1 and rituximab is administered to a patient for the duration of the patient's life.

In some embodiments, the combination of Compound 1 and rituximab is administered for at least six 28-day cycles, and Compound 1 is administered for at least one additional 28-day cycle. In some embodiments, the combination of Compound 1 and rituximab is administered for at least six 28-day cycles, and Compound 1 is administered for an additional two, three, four, five, six, seven, eight, nine, ten, eleven, twelve, thirteen or fourteen 28-day cycles. In some embodiments, the combination of Compound 1 and rituximab is administered for at least six 28-day cycles, and Compound 1 is administered for the duration of the patient's life. In some embodiments, Compound 1 is administered on days 1 through 28 (for example, one dose each day or two doses each day) of one or more 28-day cycles and rituximab is administered on day 1 of a 28-day cycle. In some embodiments, Compound 1 is administered on days 1 through 28 of one or more 28-day cycles and rituximab is administered on day 2 of a 28-day cycle.

In some embodiments, two adjacent 28-day cycles may be separated by a rest period. Such a rest period may be one, two, three, four, five, six, seven or more days during which the patient is not administered either or both Compound 1 and rituximab. In a preferred embodiment, two adjacent 28-day cycles are continuous.

In some embodiments, provided methods comprise administering to a patient in need thereof Compound 1 in combination with rituximab, wherein the patient has failed at least one prior therapy.

### Unit Dosage Forms

Pharmaceutical compositions for use in the present invention may be prepared as a unit dosage form. A person of ordinary skill will appreciate that the unit dosage forms described herein refer to an amount of a component in its free base form. A person skilled in the art will further appreciate that, when a pharmaceutical composition comprises a salt form of one component, for example, a besylate salt form of Compound 1, the amount of the salt form present in the composition is an amount that is equivalent to a unit dose of the free base of the component (i.e., of Compound 1). For example, a pharmaceutical composition comprising a besylate salt of Compound 1 would contain 34.97 mg of the besylate salt form necessary to deliver an equivalent 25 mg unit dose of the free base of Compound 1.

In some embodiments, provided methods comprise administering to a patient in need thereof a therapeutically effective amount of Compound 1, wherein the therapeutically effective amount of Compound 1 is about 250 mg to about 1250 mg. In some embodiments, the therapeutically effective amount of Compound 1 is administered as one or more discreet doses. For example, in some embodiments, a therapeutically effective amount of Compound 1 is 250 mg, wherein the therapeutically effective amount is administered as 125 mg twice daily (BID). In some embodiments, a therapeutically effective amount of Compound 1 is 500 mg, wherein the therapeutically effective amount is administered as 250 mg twice daily (BID). In some embodiments, a therapeutically effective amount of Compound 1 is 750 mg, wherein the therapeutically effective amount is administered as 375 mg twice daily (BID). In some embodiments, a therapeutically effective amount of Compound 1 is 1000 mg, wherein the therapeutically effective amount is administered as 500 mg twice daily (BID).

In some embodiments, provided methods comprise administering to a patient in need thereof a therapeutically effective amount of Compound 1, wherein the therapeutically effective amount of Compound 1 is about 125 mg to about 1250 mg, or about 125 mg to about 1125 mg, or about 125 mg to about 1000 mg, or about 125 mg to about 875 mg, or about 125 mg to about 750 mg, or about 125 mg to about 625 mg, or about 125 mg to about 500 mg, or about 125 mg to about 375 mg, or about 125 mg to about 250 mg, or about 250 mg to about 1250 mg, or about 250 mg to about 1125 mg, or about 250 mg to about 1000 mg, or about 250 mg to about 875 mg, or about 250 mg to about 750 mg, or about 250 mg to about 625 mg, or about 250 mg to about 500 mg, or about 250 mg to about 375 mg, or about 375 mg to about 1250 mg, or about 375 mg to about 1125 mg, or about 375 mg to about 1000 mg, or about 375 mg to about 875 mg, or about 375 mg to about 750 mg, or about 375 mg to about 625 mg, or about 375 mg to about 500 mg, or about 500 mg to about 1250 mg, or about 500 mg to about 1125 mg, or about 500 mg to about 1000 mg, or about 500 mg to about 750 mg, or about 500 mg to about 625 mg, or about 625 mg to about 1250 mg, or about 625 mg to about 1125 mg, or about 625 mg to about 1000 mg, or about 625 mg to about 875 mg, or about 625 mg to about 750 mg, or about 750 mg to about 1250 mg, or about 750 mg to about 1125 mg, or about 750 mg to about 1000 mg, or about 875 mg to about 1250 mg, or about 875 mg to about 1125 mg, or about 875 mg to about 1000 mg.

In some embodiments, provided methods comprise administering to a patient in need thereof a therapeutically effective amount of Compound 1, wherein the therapeutically effective amount of Compound 1 is about 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1000 mg, 1005 mg, 1010 mg, 1015 mg, 1020 mg, 1025 mg, 1030 mg, 1035 mg, 1040 mg, 1045 mg, 1050 mg, 1055 mg, 1060 mg, 1065 mg, 1070 mg, 1075 mg, 1080 mg, 1085 mg, 1090 mg, 1095 mg, 1100 mg, 1105 mg, 1110 mg, 1115 mg, 1120 mg, 1125 mg, 1130 mg, 1135 mg, 1140 mg, 1145 mg, 1150 mg, 1155 mg, 1160 mg, 1165 mg, 1170 mg, 1175 mg, 1180 mg, 1185 mg, 1190 mg, 1195 mg, 1200 mg, 1205 mg, 1210 mg, 1215 mg, 1220 mg, 1225 mg, 1230 mg, 1235 mg, 1240 mg, 1245 mg or 1250 mg.

In some embodiments, provided methods comprise administering to a patient in need thereof a pharmaceutical composition comprising a unit dose of Compound 1 in combination with rituximab. In some such embodiments, the unit dose of Compound 1 is about 25 mg, about 50 mg, about 75 mg, about 100 mg, about 125 mg, about 150 mg, about 175 mg, about 200 mg, about 225 mg or about 250 mg.

In some embodiments, provided methods comprise administering to a patient in need thereof a pharmaceutical composition comprising rituximab, wherein rituximab is administered as an infusion at a rate of 50 mg/hr. In some embodiments, the infusion rate of rituximab is increased by 50 mg/hr every 30 minutes, to a maximum of 400 mg/hr. In some embodiments, the infusion rate of rituximab is increased by 100 mg/hr every 30 minutes, to a maximum of 400 mg/hr. Accordingly, in some embodiments, the infusion rate of rituximab is 100 mg/hr. In some embodiments, the infusion rate of rituximab is 150 mg/hr. In some embodiments, the infusion rate of rituximab is 200 mg/hr. In some embodiments, the infusion rate of rituximab is 250 mg/hr. In some embodiments, the infusion rate of rituximab is 300 mg/hr. In some embodiments, the infusion rate of rituximab is 350 mg/hr. In some embodiments, the infusion rate of rituximab is 400 mg/hr.

### II. Uses of Compounds and Pharmaceutically Acceptable Compositions

Compound 1 and compositions described herein are generally useful for the inhibition of protein kinase activity of one or more enzymes. Examples of kinases that are inhibited by Compound 1 and compositions described herein and against which the methods described herein are useful include BTK and other TEC-kinases, including ITK, TEC, BMX and RLK, or a mutant thereof

Bruton's tyrosine kinase (“BTK”), a member of TEC-kinases, is a key signaling enzyme expressed in B-lymphocytes, monocytes, and mast cells or basophils. BTK plays an essential role in the B-cell signaling pathway linking cell surface B-cell receptor (BCR) stimulation to downstream intracellular responses.

BTK is a key regulator of B-cell development, activation, signaling, and survival (Kurosaki, Curr. Op. Imm., 2000, 276-281; Schaeffer and Schwartzberg, Curr. Op. Imm. 2000, 282-288). In addition, BTK plays a role in a number of other hematopoietic cell signaling pathways, e.g., Toll like receptor (TLR) and cytokine receptor-mediated TNF-α production in macrophages, IgE receptor (Fc_epsilon_RI) signaling in mast cells, inhibition of Fas/APO-1 apoptotic signaling in B-lineage lymphoid cells, and collagen-stimulated platelet aggregation. See, e.g., C. A. Jeffries, et al., (2003), Journal of Biological Chemistry 278:26258-26264; N. J. Horwood, et al., (2003), The Journal of Experimental Medicine 197: 1603-1611; Iwaki et al. (2005), Journal of Biological Chemistry 280(48):40261-40270; Vassilev et al. (1999), Journal of Biological Chemistry 274(3): 1646-1656, and Quek et al. (1998), Current Biology 8(20): 1137-1140.

Patients with inherited inactivating mutations in BTK have a profound block in B-cell development, resulting in the almost complete absence of mature B lymphocytes and plasma cells, severely reduced Ig levels and a profound inhibition of humoral response to recall antigens (reviewed in Vihinen et al Frontiers in Bioscience 5:d917-928). Mice deficient in BTK also have a reduced number of peripheral B-cells and greatly decreased serum levels of IgM and IgG3. BTK deletion in mice has a profound effect on B-cell proliferation induced by anti-IgM, and inhibits immune responses to thymus-independent type II antigens (Ellmeier et al, J Exp Med 192: 1611-1623 (2000)). BTK also plays a crucial role in mast cell activation through the high-affinity IgE receptor (Fc_epsilon_RI). BTK deficient murine mast cells have reduced degranulation and decreased production of proinflammatory cytokines following Fc_epsilon_RI cross-linking (Kawakami et al. Journal of Leukocyte Biology 65: 286-290).

Compound 1 is an inhibitor of BTK and therefore useful for treating one or more disorders associated with activity of BTK. Thus, in some embodiments, the present invention provides a method of treating, stabilizing or lessening the severity or progression of a BTK-mediated disorder comprising the step of administering to a patient in need thereof Compound 1 in combination with rituximab.

### Chronic Lymphocytic Leukemia and Small Lymphocytic Lymphoma

The B-cell disorders chronic lymphocytic leukemia (CLL) and small lymphocytic lymphoma (SLL) represent two ends of a spectrum of the same disease process differing in the degree of blood/marrow involvement (CLL) versus lymph node involvement (SLL). CLL is a lymphoproliferative malignancy characterized by progressive accumulation of morphologically mature but functionally incompetent lymphocytes in the blood, bone marrow, and lymphoid tissues. It affects mainly elderly individuals with the median age at presentation of 65 to 70 years. The clinical course of CLL ranges from indolent disease with long-term survival over 12 years to aggressive disease with median survival of 2 years.

Chronic lymphocytic leukemia is the most common leukemia in the U.S. and is typically characterized immunophenotypically as CD5+, CD23+, CD10−, CD19+, CD20 dim, sIg dim, and cyclin D1− (the latter point a distinguishing feature from mantle cell lymphoma). Chronic lymphocytic leukemia must also be distinguished from monoclonal B lymphocytosis (absolute monoclonal B-cell count<5000/μL and absence of adenopathy or other clinical features of lymphoproliferative disorder). The understanding of CLL/SLL biology and prognostic factors, and advances in formulating a risk-stratified approach to treatment of CLL/SLL have been recently reviewed by Lanasa, Furman, and the National Comprehensive Cancer Network NHL panel.

The cellular expression of Btk is restricted and largely limited to B-lymphocytes, monocytes, and mast cells or basophils. Investigation has revealed that some B-cell lymphomas and CLL/SLL depend on BCR signaling, suggesting that interruption of such signaling could be a promising therapeutic opportunity Recently it has been reported that half of all CLL retain BCR signaling in vitro and that immunoglobulin heavy gene somatic mutation (IgVH) is an important determinant of BCR responsiveness. Indeed, the mutational status of the BCR in CLL is one of the strongest predictors of disease progression, as aggressive disease typically displays BCR encoded by unmutated immunoglobulin variable heavy chains.

Two groups have reported that mutated and unmutated CLL cells respond differentially to IgM ligation of the BCR, with unmutated, but not mutated, CLL cells responding to BCR stimulation with increased global tyrosine phosphorylation and by up-regulating several genes associated with cell cycle regulation and allowing cell growth and expansion. These data highlight the differential role that BCR signaling plays in CLL physiology depending on IgVH mutational status and may suggest a possible differential responsiveness of CLL to inhibitors of BCR signaling. Other in vitro studies have reported that specific Btk inhibition with the investigational agent PCI-32765 produced substantially more apoptosis and cytotoxicity in CLL cells relative to normal B-cells; as well as inducing apoptosis in the face of anti-apoptotic micro-environmental signals, reduction of secretion of chemokines CCL3 and CCL4, and reduction of chemotaxis towards the chemokines CXCL12 and CXCL13. Detailed studies of the pathophysiologic role of Btk in the origin and/or maintenance of Waldenstrom's macroglobulinemia (WM) have not yet been reported. However, a recent report investigating transgenic mouse models demonstrated that constitutively active Btk expression resulted in selective expansion or survival of B-1 cells that were driven into germinal center independent plasma cell differentiation, as evidenced by increased numbers of IgM+plasma cells in spleen and bone marrow and significantly elevated serum IgM. Anti-nucleosome autoantibodies and glomerular IgM deposition were also observed. However, one study of sequence analysis in 19 WM patients with hypogammaglobulinemia G and/or A failed to find any novel variants in the promoter, flanking introns, or exons of Btk.

Allogeneic stem cell transplant is the only potentially curative treatment for CLL, but 70% of affected patients are ≧65 years of age at the time of diagnosis, have co-morbid conditions limiting eligibility for such therapy, and may exhibit a prolonged natural history with or without specific treatment. The actual prognosis of CLL is variable and dependent principally on clinical stage and certain genetic and molecular features. Both the Rai and Binet clinical staging systems are able to distinguish patient prognostic groups with median OSs ranging from 19 months in the most advanced stage (thrombocytopenia) to >150 months in the earliest stage (blood and marrow lymphocytosis without adenopathy, organomegaly, or defined anemia/thrombocytopenia). Classification by the presence or absence of IgVH and by interphase fluorescent in situ hybridization (iFISH) analysis for probed-for acquired chromosomal abnormalities adds additional prognostic discrimination to clinical staging, with unmutated IgVH and del(11q) and del(17p) cytogenetics predicting poorer outcome.

The CLL treatment algorithm is complex and requires first the decision to treat (e.g., presence of symptoms such as fatigue or night sweats; bulky adenopathy/organomegaly; progressive anemia/thrombocytopenia); and second, choice of the treatment regimen, usually involving one or more: purine nucleosides (fludarabine), alkylating agents (cyclophosphamide, chlorambucil, bendamustine), corticosteroids, anti-CD20 monoclonal antibodies (rituximab/ofatumumab), or anti-CD52 monoclonal antibodies (alemtuzumab). The choice of specific therapies depends on the patient's age, disease pattern (eg, primarily nodal versus non-nodal), anticipated drug tolerance and contraindications, and presence or absence of adverse prognostic features such as del(11q) or del(17p). Despite numerous therapies, treatment options are eventually limited by drug toxicities and resistance, and patients who do not succumb to other maladies endure progressive complications relating to cytopenias, the effects of lymphadenopathy and organomegaly, systemic symptoms, and infectious complications. Given the often elderly character of the patient population, an orally available, well tolerated treatment that exploits a novel weakness of CLL would be welcome.

### Rationale for Targeting Btk and Combinations with Rituximab in CLL and SLL

Strategies specifically targeting B-cells, for example the B-cell depleting anti-CD20 monoclonal antibodies rituximab and ofatumumab, have demonstrated clinical efficacy in B-cell lymphoma and CLL. Spleen tyrosine kinase (Syk) is a kinase in the BCR signaling pathway proximal to Btk. Inhibition of Syk with the orally available Syk inhibitor fostamatinib disodium produced clinical responses in DLBCL, CLL, and mantle cell lymphoma. Most tellingly, clinical proof of concept for Btk inhibition has been demonstrated by clinical investigations of the orally available Btk inhibitor PCI-32765, which have reported objective anti-tumor responses in patients with DLBCL; mantle cell, marginal zone/ mucosa-associated lymphoid tissue (MALT), and follicular lymphoma (FL), WM, and CLL/SLL, with good tolerability.

Thus, based on the critical importance of BCR signaling mediated through Btk for the survival and proliferation of various malignant B-cells; Btk's limited cellular expression in B-cells, macrophages, and monocytes; and demonstrated pre-clinical and early clinical proofs of concept that Btk inhibition produces salutary anti-lymphoma, CLL, and WM effects with acceptable clinical tolerability, targeting Btk with a selective Btk inhibitor is a promising and appropriate therapeutic strategy to investigate further in the clinic. Compound 1, as its besylate salt, has been shown in recent studies to be safe and effective against CLL as a single agent therapeutic. As of Sep. 11, 2012, 35 out of 43 patients with CLL and have experienced stable disease and continue to on treatment with N-(3-(5-fluoro-2-(4-(2-methoxyethoxy)phenylamino)pyrimidin-4-ylamino)phenyl)acrylamide besylate monotherapy. 15 of 23 patients have experienced reductions in lymph node size, and 28 of 33 patients have experienced early increases in absolute lymphocyte counts (ALC). See U.S. patent application Ser. No. 13/661,678 and International Patent Application No. PCT/US2012/062133, both filed on Oct. 26, 2012, each of which is hereby incorporated by reference in its entirety. Such data strongly support the use of a BTK inhibitor, and Compound 1 in particular, for treating CLL. Compound 1 is generally well tolerated as a single agent at up to 750 mg PO QD and the maximum tolerated dose (MTD) has not yet been reached. Studies are ongoing and additional dose levels currently being investigated include: 1000 mg QD, 1250 mg QD, 375 mg BID and 500 mg BID.

Rituximab has also been shown to exhibit good activity against relapsed/refractory CLL patients. In one study, rituximab, in combination with fludarabine/cyclophosphamide, was evaluated in 408 patients with CLL and showed an 86% response rate, as compared to the 73% response rate observed for fludarabine/cyclophosphamide alone. The median progression-free survival was 39.8 months, as compared to 31.5 months observed for fludarabine/cyclophosphamide alone. Accordingly, in some embodiments, the present invention encompasses the recognition that a BTK inhibitor such as Compound 1 in combination with rituximab is useful in the treatment of CLL and SLL. Compound 1 either as a single agent or in combination, may be found to be efficacious in CLL patients, including but not limited to those who had expressed one or more of the following prognostic/genetic markers and cytogenetic risk factors: deletions of chromosome 11q, 17p or 13q, or Trisomy 12 and 14q, zeta-chain-associated protein kinase 70 (ZAP 70) or immunoglobulin heavy chain variable region (IgVH) un-mutated.

In some embodiments, the present invention provides methods of treating, stabilizing or lessening the severity or progression of one or more diseases and conditions associated with BTK comprising administering to a patient in need thereof Compound 1 in combination with rituximab.

### III. Methods of Treating Diseases or Disorders Associated with Btk

In some embodiments, the present invention provides a method of treating, stabilizing or lessening the severity or progression of a disease or disorder selected from the group consisting of chronic lymphocytic leukemia and small lymphocytic lymphoma, the method comprising administering to a patient in need thereof Compound 1 in combination with rituximab.

In some embodiments, provided methods comprise administering to a patient in need thereof a composition comprising Compound 1 in combination with a composition comprising rituximab. In some embodiments, the composition comprising Compound 1 further comprises one or more pharmaceutically acceptable excipients. In some such embodiments, the composition comprising Compound 1 is formulated as an oral dosage form. In some embodiments, the oral dosage form is a capsule.

In some embodiments, the composition comprising rituximab further comprises one or more pharmaceutically acceptable excipients. In some such embodiments, the composition comprising rituximab is formulated as an intravenous dosage form.

In some embodiments, provided methods comprise administering to a patient in need thereof a unit dose of Compound 1 in combination with a unit dose of rituximab. In some embodiments, the unit dose of Compound 1 is about 25 mg, about 50 mg, about 75 mg, about 100 mg, about 125 mg, about 150 mg, about 175 mg, about 200 mg, about 225 mg or about 250 mg.

In some embodiments, provided methods comprise administering to a patient in need thereof a pharmaceutical composition comprising rituximab, wherein rituximab is administered as an infusion at a rate of 50 mg/hr. In some embodiments, the infusion rate of rituximab is increased by 50 mg/hr every 30 minutes, to a maximum of 400 mg/hr. In some embodiments, the infusion rate of rituximab is increased by 100 mg/hr every 30 minutes, to a maximum of 400 mg/hr. Accordingly, in some embodiments, the infusion rate of rituximab is 100 mg/hr. In some embodiments, the infusion rate of rituximab is 150 mg/hr. In some embodiments, the infusion rate of rituximab is 200 mg/hr. In some embodiments, the infusion rate of rituximab is 250 mg/hr. In some embodiments, the infusion rate of rituximab is 300 mg/hr. In some embodiments, the infusion rate of rituximab is 350 mg/hr. In some embodiments, the infusion rate of rituximab is 400 mg/hr.

In some embodiments, the present invention provides a method of treating, stabilizing or lessening the severity or progression of a disease or disorder selected from the group consisting of chronic lymphocytic leukemia and small lymphocytic lymphoma, the method comprising administering to a patient in need thereof Compound 1 in combination with rituximab, wherein the patient has failed at least one prior therapy.

In some embodiments, provided methods comprise administering to a patient in need thereof about 500 mg to about 1250 mg Compound 1 in combination with about 375 mg/m² to about 500 mg/m² rituximab. In some embodiments, provided methods comprise administering to a patient in need thereof about 750 mg to about 1000 mg Compound 1 and about 375 mg/m² to about 500 mg/m² rituximab.

In some embodiments, the present invention provides a method of treating, stabilizing or lessening the severity or progression of a disease or disorder selected from the group consisting of chronic lymphocytic leukemia and small lymphocytic lymphoma, the method comprising administering to a patient in need thereof about 375 mg BID to about 500 mg BID Compound 1 in combination with about 375 mg/m² to about 500 mg/m² rituximab. In some such embodiments, rituximab is administered once during a 28-day cycle.

In some embodiments, provided methods comprise administering to a patient in need thereof about 125 mg BID Compound 1 and about 375 mg/m² rituximab.

In some embodiments, provided methods comprise administering to a patient in need thereof about 125 mg BID Compound 1 and about 500 mg/m² rituximab.

In some embodiments, provided methods comprise administering to a patient in need thereof about 250 mg BID Compound 1 and about 375 mg/m² rituximab.

In some embodiments, provided methods comprise administering to a patient in need thereof about 250 mg BID Compound 1 and about 500 mg/m² rituximab.

In some embodiments, provided methods comprise administering to a patient in need thereof about 375 mg BID Compound 1 and about 375 mg/m² rituximab.

In some embodiments, provided methods comprise administering to a patient in need thereof about 375 mg BID Compound 1 and about 500 mg/m² rituximab.

In some embodiments, provided methods comprise administering to a patient in need thereof about 500 mg BID Compound 1 and about 375 mg/m² rituximab.

In some embodiments, provided methods comprise administering to a patient in need thereof about 500 mg BID Compound 1 and about 500 mg/m² rituximab.

In some embodiments, rituximab is administered once during a 28-day cycle. In some embodiments, rituximab is administered on cycle 1 day 2. In some embodiments, rituximab is administered on day 1 of a 28-day cycle. In some embodiments, rituximab is administered on cycle 2 day 1. In some embodiments, rituximab is administered on cycle 3 day 1. In some embodiments, rituximab is administered on cycle 4 day 1. In some embodiments, rituximab is administered on cycle 5 day 1. In some embodiments, rituximab is administered on cycle 6 day 1. In some embodiments, rituximab is administered on each of cycle 1 day 2, cycle 2 day 1, cycle 3 day 1, cycle 4 day 1, cycle 5 day 1 and cycle 6 day 1.

In some embodiments, 375 mg/m² rituximab is administered on cycle 1 day 2, and 500 mg/m² rituximab is administered on cycle 2 day 1. In some embodiments, 375 mg/m² rituximab is administered on cycle 1 day 2, and 500 mg/m² rituximab is administered on each of cycle 2 day 1 and cycle 3 day 1. In some embodiments, 375 mg/m² rituximab is administered on cycle 1 day 2, and 500 mg/m² rituximab is administered on each of cycle 2 day 1, cycle 3 day 1 and cycle 4 day 1. In some embodiments, 375 mg/m² rituximab is administered on cycle 1 day 2, and 500 mg/m² rituximab is administered on each of cycle 2 day 1, cycle 3 day 1, cycle 4 day 1 and cycle 5 day 1. In some embodiments, 375 mg/m² rituximab is administered on cycle 1 day 2, and 500 mg/m² rituximab is administered on each of cycle 2 day 1, cycle 3 day 1, cycle 4 day 1, cycle 5 day 1 and cycle 6 day 1.

In some embodiments, the combination of Compound 1 and rituximab is administered over a period of 28 consecutive days (“a 28-day cycle”). In some embodiments, the combination of Compound 1 and rituximab is administered for two, three, four, five or six 28-day cycles. In some embodiments, the combination of Compound 1 and rituximab is administered for one, two, three, four, five or six 28-day cycles, and Compound 1 is administered for an additional one, two, three, four, five, six, seven, eight, nine, ten, eleven, twelve, thirteen or fourteen 28-day cycles. In some embodiments, the combination of Compound 1 and rituximab is administered to a patient for one, two, three, four, five or six 28-day cycles, and Compound 1 is administered for the duration of the patient's life. In some embodiments, the combination of Compound 1 and rituximab is administered to a patient for one, two, three, four, five or six 28-day cycles, and either of Compound 1 or rituximab is further administered to the patient for one or more additional 28-day cycles. In some embodiments, the combination of Compound 1 and rituximab is administered to a patient for the duration of the patient's life.

In some embodiments, two adjacent 28-day cycles may be separated by a rest period. Such a rest period may be one, two, three, four, five, six, seven or more days during which the patient is not administered either or both Compound 1 and rituximab. In a preferred embodiment, two adjacent 28-day cycles are continuous.

In some embodiments, provided methods comprise administering to a patient in need thereof Compound 1 in combination with rituximab, wherein the patient has failed at least one prior therapy.

In some embodiments, the present invention provides a system for treating, stabilizing or lessening the severity of one or more diseases or conditions associated with BTK, the system comprising Compound 1 and rituximab. In some embodiments, the system is a kit. In some such embodiments, the kit comprises a pharmaceutical composition comprising Compound 1 and a pharmaceutical composition comprising rituximab.

In some embodiments, the kit comprises twenty-eight (28) daily doses of Compound 1 and one 10 mg/mL vial of rituximab. In some embodiments, the kit comprises twenty-eight (28) daily doses of Compound 1 and one 100 mg/10 mL vial of rituximab. In some embodiments, the kit comprises twenty-eight (28) daily doses of Compound 1 and one 500 mg/50 mL vial of rituximab.

In some embodiments, the kit comprises fifty-six (56) 375 mg doses of Compound 1 and one 10 mg/mL vial of rituximab. In some embodiments, the kit comprises fifty-six (56) 375 mg doses of Compound 1 and one 100 mg/10 mL vial of rituximab. In some embodiments, the kit comprises fifty-six (56) 375 mg doses of Compound 1 and one 500 mg/50 mL vial of rituximab.

In some embodiments, the kit comprises two 375 mg doses of Compound 1 and one 10 mg/mL vial of rituximab. In some embodiments, the kit comprises two 375 mg doses of Compound 1 and one 100 mg/10 mL vial of rituximab. In some embodiments, the kit comprises two 375 mg doses of Compound 1 and one 500 mg/50 mL vial of rituximab. In some embodiments, the kit comprises two 500 mg doses of Compound 1 and one 10 mg/mL dose of rituximab. In some embodiments, the kit comprises two 500 mg doses of Compound 1 and one 100 mg/10 mL vial of rituximab. In some embodiments, the kit comprises two 500 mg doses of Compound 1 and one 500 mg/50 mL vial of rituximab.

### IV. Formulations Comprising Compound 1

As described above, provided methods comprise administering to a patient in need thereof a pharmaceutically acceptable composition comprising Compound 1, wherein the pharmaceutically acceptable composition is an oral dosage form. In some embodiments, the pharmaceutically acceptable composition is formulated as a capsule.

In certain embodiments, provided methods comprise administering to a patient in need thereof a pharmaceutically acceptable composition which comprises Compound 1, and one or more pharmaceutically acceptable excipients, such as, for example, binders, film coatings, diluents, disintegrants, surfactants (wetting agents), lubricants and glidants (adsorbents), or combinations thereof. One skilled in the art will readily appreciate that the category under which a particular component is listed is not intended to be limiting; in some cases a particular component might appropriately fit in more than one category. Also, as will be appreciated, the same component can sometimes perform different functions, or can perform more than one function, in the context of a particular formulation, for example depending upon the amount of the ingredient and/or the presence of other ingredients and/or active compound(s). In some embodiments, the pharmaceutically acceptable composition is a blended powder.

### i. Binders and Diluents

Pharmaceutical compositions for use in the present invention may comprise one or more binders. Binders are used in the formulation of solid oral dosage forms to hold the active pharmaceutical ingredient and inactive ingredients together in a cohesive mix. In some embodiments, pharmaceutical compositions of the present invention comprise about 5% to about 50% (w/w) of one or more binders and/or diluents. In some embodiments, pharmaceutical compositions of the present invention comprise about 20% (w/w) of one or more binders and/or diluents. Suitable binders and/or diluents (also referred to as “fillers”) are known in the art. Representative binders and/or diluents include, but are not limited to, starches such as celluloses (low molecular weight HPC (hydroxypropyl cellulose), microcrystalline cellulose (e.g., Avicel®), low molecular weight HPMC (hydroxypropyl methylcellulose), low molecular weight carboxymethyl cellulose, ethylcellulose), sugars such as lactose (i.e. lactose monohydrate), sucrose, dextrose, fructose, maltose, glucose, and polyols such as sorbitol, mannitol, lactitol, malitol and xylitol, or a combination thereof. In some embodiments, a provided composition comprises a binder of microcrystalline cellulose and/or lactose monohydrate.

### ii. Disintegrants

Pharmaceutical compositions for use in the present invention may further comprise one or more disintegrants. Suitable disintegrants are known in the art and include, but are not limited to, agar, calcium carbonate, sodium carbonate, sodium bicarbonate, cross-linked sodium carboxymethyl cellulose (croscarmellose sodium), sodium carboxymethyl starch (sodium starch glycolate), microcrystalline cellulose, or a combination thereof. In some embodiments, provided formulations comprise from about 1%, to about 25% disintegrant, based upon total weight of the formulation.

### iii. Surfactants

Surfactants, also referred to as bioavailability enhancers, are well known in the art and typically facilitate drug release and absorption by enhancing the solubility of poorly-soluble drugs. Representative surfactants include, but are not limited to, poloxamers, polyoxyethylene ethers, polyoxyethylene fatty acid esters, polyethylene glycol fatty acid esters, polyoxyethylene hydrogenated castor oil, polyoxyethylene alkyl ether, polysorbates, and combinations thereof. In certain embodiments, the surfactant is a poloxamer. In some such embodiments, the poloxamer is poloxamer 407. In some embodiments, compositions for use in the present invention comprise from about 1% to about 30% by weight of surfactant, based upon total weight of the blended powder.

### iv. Lubricants

Pharmaceutical compositions of the present invention may further comprise one or more lubricants. Lubricants are agents added in small quantities to formulations to improve certain processing characteristics. Lubricants prevent the formulation mixture from sticking to the compression machinery and enhance product flow by reducing interparticulate friction. Representative lubricants include, but are not limited to, magnesium stearate, glyceryl behenate, sodium stearyl fumarate and fatty acids (i.e. palmitic and stearic acids). In certain embodiments, a lubricant is magnesium stearate. In some embodiments, provided formulations comprise from about 0.2% to about 3% lubricant, based upon total weight of given formulation.

### v. Glidants

Pharmaceutical compositions of the present invention may further comprise one or more glidants. Representative glidants include, but are not limited to, silicas (i.e. fumed silica), microcrystalline celluloses, starches (i.e. corn starch) and carbonates (i.e. calcium carbonate and magnesium carbonate). In some embodiments, provided formulations comprise from about 0.2% to about 3% glidant, based upon total weight of given formulation.

### vi. N-(3-(5-fluoro-2-(4-(2-methoxyethoxy)phenylamino)pyritnidin-4-ylamino)phenyl)acrylamide besylate

As described above, the present invention provides a method of treating a disease or disorder selected from chronic lymphocytic leukemia and small lymphocytic lymphoma, the method comprising administering to a patient in need thereof Compound 1 in combination with rituximab. The besylate salt of Compound 1, N-(3-(5-fluoro-2-(4-(2-methoxyethoxy)phenylamino)pyrimidin-4-ylamino)phenyl)acrylamide benzenesulfonic acid salt, has recently been identified and is currently in clinical trials as monotherapy in subjects with relapsed or refractory B-cell non-Hodgkin's lymphoma (B-NHL), chronic lymphocytic leukemia (CLL) and Waldenstrom's macroglobulinemia (WM). Thus, in some embodiments, provided methods comprise administering to a patient in need thereof a besylate salt of Compound 1.

In some embodiments, provided methods comprise administering to a patient in need thereof a pharmaceutically acceptable composition comprising from about 5% to about 60% of the besylate salt of Compound 1, based upon total weight of the formulation. In some embodiments, provided methods comprise administering to a patient in need thereof a pharmaceutically acceptable composition comprising from about 5% to about 15% or about 7% to about 15% or about 7% to about 10% or about 9% to about 12% of the besylate salt of Compound 1, based upon total weight of the composition. In some embodiments, provided methods comprise administering to a patient in need thereof a pharmaceutically acceptable composition comprising from about 25% to about 75% or about 30% to about 60% or about 40% to about 50% or about 40% to about 45% of the besylate salt of Compound 1, based upon total weight of the formulation. In certain embodiments, provided methods comprise administering to a patient in need thereof a pharmaceutically acceptable composition comprising from about 8%, about 9%, about 10%, about 11%, about 12%, about 13%, about 20%, about 30%, about 40%, about 41%, about 42%, about 43%, about 44%, about 45%, about 50%, about 60%, about 70%, or about 75% of the besylate salt of Compound 1, based upon total weight of given composition or formulation.

In some such embodiments, provided methods comprise administering to a patient in need thereof a pharmaceutical composition comprising a unit dose of Compound 1, wherein Compound 1 is in the form of a besylate salt. In some such embodiments, the unit dose is an amount sufficient to provide about 25 mg, about 50 mg, about 75 mg, about 100 mg, about 125 mg, about 150 mg, about 175 mg, about 200 mg, about 225 mg or about 250 mg of the free base of Compound 1. In some embodiments, the pharmaceutical composition comprising the besylate salt of Compound 1 is a solid oral dosage form.

In some embodiments, the present invention provides a method of treating, stabilizing or lessening the severity or progression of a disease or disorder selected from the group consisting of chronic lymphocytic leukemia and small lymphocytic lymphoma, the method comprising administering to a patient in need thereof Compound 1 in combination with rituximab, wherein Compound 1 is administered as the besylate salt. In some such embodiments, the besylate salt of Compound 1 is administered in the form of a composition comprising one or more pharmaceutically acceptable excipients selected from binders, film coatings, diluents, disintegrants, surfactants, lubricants and glidants.

In some embodiments, the present invention provides a method of treating, stabilizing or lessening the severity or progression of a disease or disorder selected from the group consisting of chronic lymphocytic leukemia and small lymphocytic lymphoma, the method comprising administering to a patient in need thereof a pharmaceutical composition comprising the besylate salt of Compound 1 in combination with rituximab, wherein the amount of besylate salt of Compound 1 is sufficient to deliver about 125 mg, about 250 mg, about 325 mg, about 375 mg, about 400 mg, about 500 mg, about 625 mg, about 750 mg, about 1000 mg or about 1250 mg of the free base of Compound 1. In some such embodiments, the pharmaceutical composition further comprises one or more pharmaceutically acceptable excipients selected from binders, film coating, diluents, disintegrants, surfactants, lubricants and glidants. In some such embodiments, the pharmaceutical composition comprises one or more pharmaceutically acceptable excipients selected from microcrystalline cellulose, lactose monohydrate, sodium starch, poloxamer 407, fumed silica and magnesium stearate.

### V. Process for Preparing Pharmaceutical Compositions Comprising Compound 1 Dry Blend Process

Milled N-(3-(5-fluoro-2-(4-(2-methoxyethoxy)phenylamino)pyrimidin-4-ylamino)phenyl)acrylamide besylate, milled microcrystalline cellulose, milled sodium starch glycolate, milled lactose monohydrate, milled poloxamer 407, and sieved fumed silica are weighed and mechanically blended. An intragranular portion of sieved magnesium stearate (2.0%, per Table 1, below) is added to the blender and the formulation blended. This blended formulation is then roller compacted, milled, and then blended. The blended formulation is additionally roller compacted, milled and then blended. The remainder or extragranular portion of the magnesium stearate (0.5%, per Table 1, below) is added and the final formulation is blended. Capsules are either mechanically filled or manually filled via the flood fill method.

All features of each of the aspects of the invention apply to all other aspects mutatis mutandis.

In order that the invention described herein may be more fully understood, the following examples are set forth. It should be understood that these examples are for illustrative purposes only and are not to be construed as limiting this invention in any manner.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Example 1

### Dose Escalation Study

N-(3-(5-fluoro-2-(4-(2-methoxyethoxy)phenylamino)pyrimidin-4-ylamino)phenyl)acrylamide besylate is a chemically synthesized small molecule substituted pyrimidine developed as the benzenesulfonic acid salt and is a white to off-white crystalline powder. N-(3-(5-fluoro-2-(4-(2-methoxyethoxy)phenylamino)pyrimidin-4-ylamino)phenyl)acrylamide besylate is an oral, potent (IC₅₀<0.5 nM) and selective small molecule inhibitor of Btk. N-(3-(5-fluoro-2-(4-(2-methoxyethoxy)phenylamino)pyrimidin-4-ylamino)phenyl)acrylamide besylate exhibits solubility of approximately 0.16 mg/mL in water and a maximum aqueous solubility of 0.40 mg/mL at approximately pH 3.0. The solubility of N-(3-(5-fluoro-2-(4-(2-methoxyethoxy)phenylamino)pyrimidin-4-ylamino)phenyl)acrylamide besylate in ethanol is approximately 10 mg/mL. N-(3-(5-fluoro-2-(4-(2-methoxyethoxy)phenylamino)pyrimidin-4-ylamino)phenyl)acrylamide besylate exhibits no environmental instabilities (i.e. heat, acid, base) that require special handling.

N-(3-(5-fluoro-2-(4-(2-methoxyethoxy)phenylamino)pyrimidin-4-ylamino)phenyl)acrylamide besylate was formulated into capsules containing the components and quantities listed in Table 1 to obtain the study drug. The capsules listed in Table 1 will be administered during the dose escalation and expansion cohort studies.

TABLE 1
Components of N-(3-(5-fluoro-2-(4-
(2-methoxyethoxy)phenylamino)pyrimidin-4-ylamino)phenyl)acrylamide
besylate capsules

| | Amount per | |
|---|---|---|
| | 125 mg | |
| Component | Capsule | |
| Capsule shell | 1, size 0 | |
| | white capsule | |
| N-(3-(5-fluoro-2-(4-(2- | 174.30 | mg |
| methoxyethoxy)phenylamino) | (125 mg free | |
| pyrimidin-4-ylamino)phenyl) | base) | |
| acrylamide besylate | | |
| Microcrystalline cellulose | 101.68 | mg |
| Lactose monohydrate | 41.50 | mg |
| Sodium starch glycolate | 41.50 | mg |
| Poloxamer 407 | 41.50 | mg |
| Fumed silica | 4.15 | mg |
| Magnesium stearate | 10.38 | mg^{‡} |

^{‡}2.0% (8.30 mg) intragranular; 0.5% (2.08 mg) extragranular.

Rituximab is provided to the physician/investigator in 10 mg/mL vials comprising 100 mg/10 mL or 500 mg/50 mL. Prior to administration, rituximab is diluted to a dose of 1 mg/mL, 2 mg/mL, 3 mg/mL or 4 mg/mL with either 5% dextrose in water or 0.9% sodium chloride. Rituximab is thereafter administered as a 1 mg/mL to 4 mg/mL infusion according to the dosages set forth in Table 2, below.

### Study Design

Subjects with relapsed or refractory CLL or SLL who failed at least one prior treatment regimen will be enrolled in a “3+3” dose escalation and expansion study to determine the Not Tolerated Dose (NTD), the Optimal Biologic Effect dose (OBE) and the Maximum Tolerated Dose (MTD) of the combination of Compound 1 and rituximab. Approximately 30-42 patients will be enrolled in the study.

Study treatment will be administered in 28-day cycles at specified dose levels as scheduled until disease progression, unacceptable toxicity, or discontinuation for any other reason. Subjects continue on the starting dose until the preliminary recommended Phase 2 dose (RP2D) is determined, at which point they can be switched to the preliminary RP2D.

N-(3-(5-fluoro-2-(4-(2-methoxyethoxy)phenylamino)pyrimidin-4-ylamino)phenyl)acrylamide besylate will be administered according to the cohorts listed in Table 2:

TABLE 2
Study Dosing Schema for Escalating Dose Portion of Study

| | N-(3-(5-fluoro-2-(4-(2- | |
|---|---|---|
| | methoxyethoxy)phenylamino) | |
| CO- | pyrimidin-4-ylamino)phenyl) | |
| HORT | acrylamide besylate | Rituximab |
| 1 | 375 mg BID | 6 doses administered as follows: |
| 2 | 500 mg BID | 375 mg/m² on cycle 1 day 2 |
| 3 | 500 mg BID | 500 mg/m² on cycles 2-6 day 1 |

Within each cohort, subjects will be treated PO (oral) BID (daily) with N-(3-(5-fluoro-2-(4-(2-methoxyethoxy)phenylamino)pyrimidin-4-ylamino)phenyl) twice acrylamide besylate during an initial 28-day treatment cycle and will be assessed for safety, tolerability and DLT, as well as pharmacokinetic (“PK”), pharmacodynamic (“PD”), and disease response. In certain instances, the physician-investigator may elect to rest a patient during the study, during which time the patient does not receive treatment. For example, the physician-investigator may elect to rest a patient due to occurrence or recurrence of adverse events. For purposes of clarity, a patient who has been rested is still enrolled in the study until the physician-investigator determines that the patient should not continue treatment, at which time such patients are discontinued from further treatment. In this context, treatment duration refers to the time a patient is enrolled in the study, inclusive of all rest periods, until treatment is discontinued.

Rituximab will be administered as a single intravenous (IV) infusion. The initial infusion during cycle 1 will be administered at 375 mg/m²; subsequent infusions during cycles 2 through 6 will be administered at 500 mg/m². Administration of rituximab will begin on day 2 of cycle 1 and on day 1 of each cycle thereafter. Following the cycle 6 infusion, rituximab will be discontinued. Subjects may continue on treatment with N-(3-(5-fluoro-2-(4-(2-methoxyethoxy)phenylamino)pyrimidin-4-ylamino)phenyl)acrylamide besylate if appropriate. The first infusion of rituximab will be at a rate of 50 mg/hr. In the absence of infusion toxicity, the infusion rate will be increased by 50 mg/hr increments every 30 minutes, to a maximum of 400 mg/hr. Each subsequent infusion will be initiated at 100 mg/hr. In the absence of infusion toxicity, the infusion rate will be increased by 100 mg/hr increments at 30 minute intervals to a maximum of 400 mg/hr.

The dose level at which a patient is enrolled will be based on which cohort is open at the time of enrollment.

Three patients will be enrolled at dose level 1 and treated with N-(3-(5-fluoro-2-(4-(2-methoxyethoxy)phenylamino)pyrimidin-4-ylamino)phenyl) acrylamide besylate BID in combination with rituximab for 28 days. Dose escalation, via enrollment in the next higher dose, will be allowed only if none (0) of the first three (3) enrolled subjects experience dose limiting toxicity (DLT). If one (1) of the first three (3) subjects dosed experiences a DLT, three (3) more subjects will be enrolled in that dose cohort. A dose level will be considered to be below the NTD if <1 of 3 DLT evaluable subjects enrolled experiences a DLT. A dose will be considered a NTD when two (2) of six (6) DLT-evaluable subjects in that cohort experience a DLT. A MTD will be declared when at least six (6) subjects have been enrolled and safely complete cycle 1 at that dose level. The MTD is defined as the last dose below the NTD with zero (0) or one (1) DLT-evaluable subjects experiencing DLT during the first 28-day cycle.

During the dose escalation phase, a decision to enroll the next higher dose cohort will be based on review of safety and DLT-evaluable patients. The OBE dose is defined as follows:
a reduction of ≧50% in the size of lymph nodes in ≧two (2) of six (6) subjects; and/or
no further increase in exposure with increasing doses; and/or
a ≧25% increase in lymphocytosis in four (4) of six (6) subjects during the first three 28-day cycles not assessed as progressive disease.

After full enrollment of each dose escalation cohort and completion of the second cycle of treatment for each dose escalation cohort, the number and type of DLTs and adverse events (AE) occurring during the first two cycles will be evaluated. Subjects will remain on study until the subject discontinues due to disease progression, unacceptable toxicity, withdrawal of consent or any other reason determined by the physician. Preliminary evidence of efficacy will be evaluated.

Expansion Cohorts. After completion of observation for DLTs in the dose escalation study, the accumulated safety, PK, and PD data will be evaluated to select a preliminary RP2D. The preliminary RP2D will be evaluated in expanded cohorts of 24 subjects or a more complete safety profile and further preliminary evaluation of efficacy. If less than 9 of 24 subjects experience DLTs, then this will be declared the RP2D to be used in further studies. If DLTs are experienced in greater than or equal to 9 of 24 subjects, this dose will be considered to have exceeded the MTD and the previous highest tolerated dose found in the dose escalation cohort of the study will be evaluated in 24 subjects. The dose level will continue to be reduced in a stepwise fashion until less than 9 of 24 subjects experience DLTs.

During the expansion cohort, rituximab will be administered according to the schedule set forth in the dose escalation cohorts. Rituximab will be administered as a single intravenous (IV) infusion. The initial infusion during cycle 1 will be administered at 375 mg/m²; subsequent infusions during cycles 2 through 6 will be administered at 500 mg/m². Administration of rituximab will begin on day 2 of cycle 1 and on day 1 of each cycle thereafter. Following the cycle 6 infusion, rituximab will be discontinued. Each subject will continue on treatment with N-(3-(5-fluoro-2-(4-(2-methoxyethoxy)phenylamino)pyrimidin-4-ylamino)phenyl)acrylamide besylate until disease progression, unacceptable toxicity or treatment discontinuation for any other reason.

In certain instances, the physician-investigator may elect to rest a patient during the study, during which time the patient does not receive treatment. For example, the physician-investigator may elect to rest a patient due to occurrence or recurrence of adverse events. For purposes of clarity, a patient who has been rested is still enrolled in the study until the physician-investigator determines that the patient should not continue treatment, at which time such patients are discontinued from further treatment. In this context, treatment duration refers to the time a patient is enrolled in the study, inclusive of all rest periods, until treatment is discontinued.

Adverse Events. For all cohorts, dose limiting toxicities (DLTs) are defined as specified adverse events (AEs) that are observed within the first two 28-day cycles (approximately 56 days) and deemed to be related to treatment. Hematologic DLTs include Grade 4 anemia (hemoglobin decrease) or thrombocytopenia by NCI-CTCAE (v. 4.03) or by IWCLL criteria, whichever results in the lower blood threshold; Grade 4 neutropenia greater than 5 days despite granulocyte colony-stimulating factor (G-CSF) support; and Grade 3 or higher febrile neutropenia. Lymphocytosis may be observed as a consequence of disease progression but has also been described as a redistribution (lymphocytle migration and trafficking) phenomenon in subjects receiving another BTK inhibitor even as lymph node disease responds to treatment. Therefore, lymphocytosis will not be rated for DLT. Reduction of malignant lymphocytosis is an intended therapeutic effect of treatment and will not be considered for DLT.

Non-hematologic DLTs include Grade 4 or higher non-hematologic AEs of any duration; Grade 3 total bilirubin elevation, whether symptomatic or asymptomatic; and any Grade 3 non-hematologic toxicity except nausea, vomiting and diarrhea lasting less than 24 hours following medical therapy; tumor lysis syndrome which does not progress to Grade 4 and resolves in less than 7 days with medical management; and transient, and Grade 3 non-hematologic laboratory anomaly that is asymptomatic and rapidly reversible (returns to baseline or ≦Grade 1 within 7 days).

Subjects without disease progression and without DLT at the end of the first 28-day cycle of treatment will be eligible to continue receiving N-(3-(5-fluoro-2-(4-(2-methoxyethoxy)phenylamino)pyrimidin-4-ylamino)phenyl)acrylamide besylate in combination with rituximab for additional 28-day cycles until (i) the patient experiences unacceptable toxicity, (ii) the underlying malignancy progresses, (iii) the patient withdraws consent, or (iv) the treating physician-investigator otherwise determines that the patient should not continue treatment.

Btk Occupancy. The covalent mechanism of action of N-(3-(5-fluoro-2-(4-(2-methoxyethoxy)phenylamino)pyrimidin-4-ylamino)phenyl)acrylamide besylate allows for development of a covalent probe to detect free, uninhibited Btk in lysates derived from tissue culture, animal tissues, or clinical samples. PBMC lysates are isolated from whole blood samples 30 minutes before dosing, 4 hours or 24 hours post-dose and incubated with the biotinylated covalent probe. Uninhibited Btk is captured by the covalent probe and quantitated by ELISA. Normalization to untreated control sample allows for determination of the % Btk occupancy.

Btk Target Site Occupancy ELISA: Cell lysates or spleen homogenates are incubated with 1 μM N¹-(3-(3-(4-(3-acrylamidophenylamino)-5-methylpyrimidin-2-ylamino)phenoxy)propyl)-N⁵-(15-oxo-19-((3aS,4S,6aR)-2-oxohexahydro-1H-thieno[3,4-d]imidazol-4-yl)-4,7,10-trioxa-14-azanonadecyl)glutaramide (2) in a PBS, 0.05% Tween-20, 1% BSA solution for 1 h at room temperature. Compound 2 has the following structure:

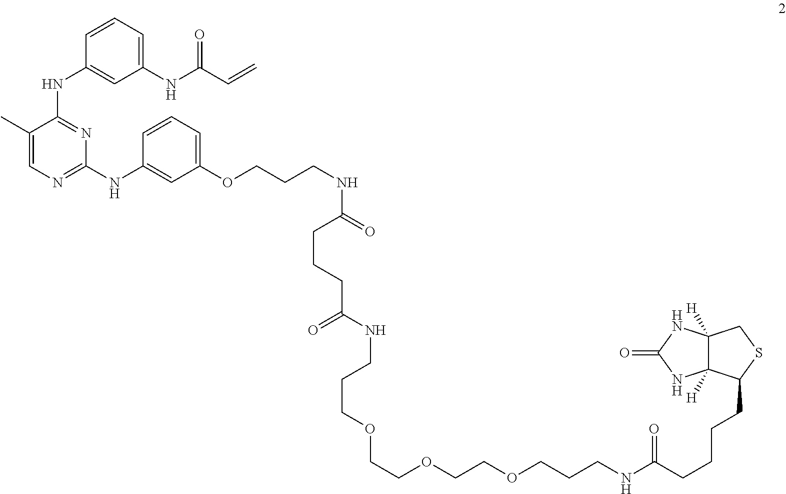 [SMILES: C=CC(=O)Nc1cccc(Nc2nc(Nc3cccc(OCCCNC(=O)CCCC(=O)NCCCOCCOCCOCCCNC(=O)CCCC[C@@H]4SC[C@@H]5NC(=O)N[C@@H]54)c3)ncc2C)c1]

Standards and samples are transferred to a streptavidin-coated 96-well ELISA plate and mixed while shaking for 1 h at room temperature. The α-Btk antibody (BD 611116, 1:1000 dilution in PBS+0.05% Tween-20+0.5% BSA) is then incubated for 1 h at room temperature. After wash, goat α-mouse-HRP (1:5000 dilution in PBS+0.05% Tween-20+0.5% BSA) is added and incubated for 1 h at room temperature. The ELISA is developed with addition of tetramethyl benzidine (TMB) followed by Stop Solution and read at OD 450 nm. The standard curve (11.7-3000 pg/μL) is generated with human full-length recombinant Btk protein and plotted using a 4 parameter curve fit in Gen5 software. Uninhibited Btk detected from samples is normalized to μg total protein as determined by BCA protein analysis (Pierce Cat. 23225).

We claim:

1. A method of treating, stabilizing or lessening the severity or progression of one or more diseases and conditions associated with BTK comprising administering to a patient in need thereof Compound 1 (N-(3-(5-fluoro-2-(4-(2-methoxyethoxy)phenylamino)pyrimidin-4-ylamino)phenyl)acrylamide): 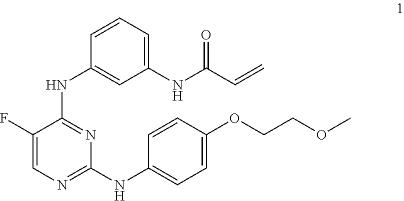 [SMILES: C=CC(=O)Nc1cccc(Nc2nc(Nc3ccc(OCCOC)cc3)ncc2F)c1]
or a pharmaceutically acceptable salt thereof, and an anti-CD20 antibody.

2. The method according to claim 1, wherein the disease or condition associated with BTK is selected from chronic lymphocytic leukemia and small lymphocytic lymphoma.

3. The method according to claim 2, wherein the patient has failed at least one prior therapy.

4. The method according to claim 2, wherein Compound 1 is administered twice a day.

5. The method according to claim 4, wherein Compound 1 is in the form of a benzenesulfonic acid salt.

6. The method according to claim 5, wherein Compound 1 is administered in the form of a composition comprising from about 10% to about 50% N-(3-(5-fluoro-2-(4-(2-methoxyethoxy)phenylamino)pyrimidin-4-ylamino)phenyl)acrylamide besylate.

7. The method according to claim 6, wherein the composition comprises about 42% N-(3-(5-fluoro-2-(4-(2-methoxyethoxy)phenylamino)pyrimidin-4-ylamino)phenyl)acrylamide besylate.

8. The method according to claim 5, wherein Compound 1 is administered as an oral dosage form.

9. The method according to claim 2, wherein the anti-CD20 antibody is rituximab.

10. The method according to claim 9, wherein rituximab is administered once during a 28-day cycle.

11. The method according to claim 10, wherein rituximab is administered as an intravenous infusion.

12. The method according to claim 9, wherein each of Compound 1 and rituximab is administered for at least one 28-day cycle.

13. A method of preventing, treating, stabilizing or lessening the severity or progression of a disease or disorder selected from the group consisting of chronic lymphocytic leukemia and small lymphocytic lymphoma, the method comprising administering to a patient in need thereof a therapeutically effective amount of Compound 1, or a pharmaceutically acceptable salt thereof, and a therapeutically effective amount of an anti-CD20 antibody, wherein the therapeutically effective amount of Compound 1 is about 750 mg to about 1000 mg per day.

14. The method according to claim 13, wherein the therapeutically effective amount of Compound 1 is about 375 mg BID.

15. The method according to claim 13, wherein the therapeutically effective amount Compound 1 is about 500 mg BID.

16. The method according to claim 13, wherein the anti-CD20 antibody is rituximab.

17. The method according to claim 16, wherein the therapeutically effective amount of rituximab is about 375 mg/m².

18. The method according to claim 16, wherein the therapeutically effective amount of rituximab is about 500 mg/m².

19. A system for treating, stabilizing or lessening the severity of one or more diseases or conditions associated with BTK, the system comprising Compound 1, or a pharmaceutically acceptable salt thereof, and an anti-CD20 antibody.

20. The system according to claim 19, wherein the anti-CD20 antibody is rituximab.
